(19) United States
(12) Patent Application Publication
(10) Pub. No.: US 2008/0152598 A1
(43) Pub. Date: Jun. 26, 2008

(54) ORAL COMPOSITION FOR STABILIZATION, (RE)CALCIFICATION AND (RE)MINERALIZATION OF TOOTH ENAMEL AND DENTINE

(75) Inventor: Robert Basic, Zagreb (HR)

Correspondence Address: KATTEN MUCHIN ROSENMAN LLP, 575 MADISON AVENUE, NEW YORK, NY, 10022-2585, US

(73) Assignee: PRODUCTA LTD., Zagreb (HR)

(21) Appl. No.: 12/042,493

(22) Filed: Mar. 5, 2008

Related U.S. Application Data
(63) Continuation-in-part of application No. 11/252,353, filed on Oct. 17, 2005, now abandoned.
Application No. 11/252,353 is a continuation of application No. PCT/HR2004/000010, filed on Apr. 15, 2004.

(30) Foreign Application Priority Data
Apr. 17, 2003 (HR) P20030304A

(51) Int. Cl. A61K 9/68; A61K 8/18; A61K 8/24

(52) U.S. Cl. 424/48; 424/49; 424/57

(57) ABSTRACT

An oral composition for the stabilization, recalcification and remineralization of dental enamel, providing efficient protection from tooth decay. The oral composition uses the calcium form of zeolite, phosphate salts soluble in water, and matrix proteins of teeth. The efficiency of this solution is based on the adjustment of pH in the mouth cavity to the required value while at the same time incorporating the calcium ions from the calcium form of zeolite into the dental enamel and dentin in the presence of matrix proteins of the teeth. Calcium and phosphate ions stabilizes the crystal structure of calcium hydroxyapatite in tooth enamel and dentin.

### RELATED APPLICATIONS

This application is a Continuation-in-Part of U.S. application Ser. No. 11/252,353, now pending, which is a continuation of International Application PCT/HR2004/000010, filed Apr. 15, 2004, now expired, which claims priority from HR P20030304A, filed Apr. 17, 2003, which is incorporated herein by reference.

### FIELD OF THE INVENTION

The present invention refers to an oral composition for prevention of tooth caries. More particularly, the invention relates to compositions and methods for stabilization, recalcification and remineralization of tooth enamel and dentin.

### DISCUSSION OF THE BACKGROUND INFORMATION

Tooth, similarly to other mineralized tissues, is liable to chemical and physical damages in places “impoverished” by calcium and “enriched” by carbonates. Since the mineral part of tooth consists of sparingly soluble mineral materials, a main reason for chemical damage of tooth is dissolution of tooth enamel in an acidic environment saturated with the components of mineral materials. Impurities, such as sodium, potassium, magnesium, lead, strontium, barium, and particularly, carbonate ions, cause damage of hydroxyapatite crystals and increase their solubility. Tooth enamel damage from demineralization or dissolution is accelerated by the action of different endogenic and exogenic factors such as pregnancy, old age, infancy, osteoporosis, progressive gum disease and gingivitis, all of which can cause dental lesions.

The mechanism of dental caries formation is essentially straightforward; plaque on the surface of tooth consists of bacteria which produces acids as a byproduct of its metabolism. Any fermentable carbohydrate such as glucose, sucrose, fructose or cooked starch can be metabolized by the acidogenic bacteria and create the aforementioned organic acids as byproducts. The formed acids diffuse through the plaque into the porous subsurface parts of enamel and dentin. The hydrogen ions formed by dissociation of organic acids dissolve the mineral part of enamel and dentin. This process is known as demineralization. Since dissolution of the mineral part of tooth is favored in an acidic environment, the process of demineralization is promoted by strong, stable acids, which are found in acidic foods, such as tomatoes or oranges. The demineralization process continues each time carbohydrates are taken into the mouth. If the demineralization process is not halted by a decrease of acidity in the mouth cavity, caries can develop.

Abundant, well documented long-term investigations have shown a positive effect of fluoride on stabilization of tooth enamel and caries prevention (J. M. ten Cate and C. van Loveren, Cariology 43 (1999) 713). The positive effect of fluoride in the prevention of tooth caries can be explained by three fundamental mechanisms (J. D. B. Featherstone, Comm. Dent. Oral Epidemiol. 27 (1999) 31):

(1) Exchange of OH⁻ ions in hydroxyapatite (Ca₅(PO₄)₃0H) by F- ions, i.e.,

Ca₅(P0₄)₃0H+F⁻ [symbol] Ca₅(PO₄)₃F+OH⁻

and by formation of flurapatite (Ca₅(PO₄F) with a solubility in an acidic environment about ten times lower than the solubility of hydroxyapatite. This stabilizes enamel and dentin by slowing down the demineralization process (J. D. B. Featherstone, R. Glena, M. Shariati and C P. Shields, J. Dent. Res. 69 (1990) 20.; J. M. ten Cate and J. D. B. Featherstone, Crit. Rev. Oral Biol. 2 (1991) 283).
(2) Enhancing remineralization on the surface by acceleration of the processes of crystallization of hydroxyapatite and fluorapatite.
(3) Inhibiting the growth of cariogenic bacteria by collecting HF in their cells. Investigations have shown that F ions which exist in neutral and alkaline media cannot pass the walls and membrane of cells, but HF, which exists in acidic medium, easily passes the cell walls and membrane of cells (G. M. Whitford, G. S. Schuster and H. D. Pashley, Infect. Immun. 18 (1977) 680; C. Van Louveren, J. Dent. Res. 69 (1990) 676; I. R. Hamilton and G. W. H. Bowden in: 0. Fjereskov, J. Ekstrans and B. A. Burt (eds.), Fluoride in Dentistry, Munksgaard, Copenhagen, 1996, p. 230). Development of caries increases the acidity of medium causing formation of HF in the presence of F⁻ ions. Formed HF collects in cells, and stops their further growth. Due to the effect of fluorine, fluorine compounds are widely used in dental medicine, as evidenced in numerous scientific publications (J. M. ten Cate and C. van Loveren, Cariology 43 (1999) 713 and J. D. B. Featherstone, Comm. Dent. Oral Epidemiol. 27 (1999) 31) and patents (for e.g., K. Brigham and R. C. Vickerv, U.S. Pat. No. 3,647,488, 1972;. Tomlinson and E. J. Duff, U.S. Pat. No. 4,048,300; M. C. S. Gaffar and A. Gaffar, U.S. Pat. No. 4,177,258; M. C. S. Gaffar and A. Gaffar, U.S. Pat. No. 4,183,915; J. Weststrate and E. M. Staal, U.S. Pat. No. 4,460,565; W. Schmidt, R. Purrmann, P. Jochum and H. J. Huebner, U.S. Pat. No. 4,472,836; J. J. Paran, Jr. and N. Y. Sakkab, U.S. Pat. No. 4,515,772; N. Usen and A. E. Winston, U.S. Pat. No. 5,605,675; A. E. Winston and N. Usen, U.S. Pat. No. 5,817,296; A. E. Winston and N. Usen, U.S. Pat. No. 5,858,333; N. Usen and A. E. Winston, U.S. Pat. No. 5,895,641; R.-R. Miethke and H. Neweseiy, German Patent DE 3,404,827; T. Reetz, S. Zimmer and W. Krahl, German Patent DE 19,735,929; J. W. Stansburry, J. M. Antonucci and K. M. Choi, U.S. Pat. No. 6,184,339; F. Rueggeberg, G. Whitford and D. Mettenburg, US Patent Appl. Publ. 2002028856, 2002), each of which is incorporated by reference in their entirety herein.

It is also known that remineralization by fluorine is effective only in the presence of calcium and phosphate ions (A. Papas, D. Russell, M. Singh, K. Stack, R. Kent, C. Triol, et al, Gerodontol 16 (2000) 2). Unfortunately, there are growing concerns connected with negative effects of fluorine on human health. (M. S. Tung and F. C. Eichmiller, J. Clin. Dent. 10(1999)1). Therefore, there remains a need to develop new approaches for prevention of caries and processes of remineralization.

U.S. Pat. No. 3,934,267 to Randel entitled “Method for remineralizing and immunizing tooth enamel for the prevention and control of tooth decay and dental caries” discusses a method based on the acidic treatment of enamel to remove positively charged calcium, which causes the formation of a porous sponge-like negatively charged surface. The enamel surface treated with a solution of positively charged heavy metal ions, depose on the negatively charged surface of enamel by electrostatic forces. In addition, tooth enamel containing heavy metals on the surface when treated with sulfur compounds forms heavy metal sulfides which are resistant to acids formed during development of caries, thereby protecting teeth against decay.

U.S. Pat. No. 4,048,300 to K. Tomlinson and E. J. Duuf entitled “Dental preparation containing materials having calcium and phosphate components” describes a cream preparation for remineralization of tooth enamel containing fluorapatite, fluorhydroxyapatite and hydroxyapatite, and materials containing monofluorphosphate, and carbonate or two-valent ions such as ZnF²₄. U.S. Pat. No. 4,080,440 to D. N. DiGiulio and R. J. Grabenstetter entitled “Method for remineralizing tooth enamel” discusses a method based on the treatment of tooth enamel with a metastable water solution of 0.005%-5% calcium and 0.005%-5% phosphate ions with the molar ratio of Ca:P between 0.01 and 100 and a pH between 2.5-4.

The solution can be used 5 minutes after preparation with a duration of application in the mouth cavity of between 10 seconds and 3 minutes, i.e. the time interval during the solution is metastable. Remineralization occurs by incorporation of calcium and phosphate ions from the solution in the demineralized surfaces of teeth.

R.J. Grabenstetter and J. A. Gray (U.S. Pat. No. 4,083,955: Processes and compositions for remineralization of dental enamel) discusses a method of remineralization in two stages. In the first stage, the mouth cavity is treated for 10-30 seconds with a 0.005-10% water solution of soluble calcium ions or soluble phosphate ions. During the treatment, calcium or phosphate ions build into surface and subsurface parts of tooth enamel. Where the mouth cavity is treated by calcium ions in the first stage, the mouth cavity is treated with phosphate ions in the second stage for the same time, and vice versa. During the second stage of the treatment, phosphate ions from the solution react with the calcium ions previously built into enamel, and the calcium ions from the solution react with the phosphate ions previously built into enamel, respectively, forming hydroxyapatite in both the cases.

M. C. S. Gaffar and A. Gaffar (U.S. Pat. No. 4,177,258: Dentifrice for dental remineralization) discusses a preparation for nursing teeth containing a source of calcium ions, i.e. a water solution containing 50 ppm, phosphate ions, i.e. a water solution containing 50 ppm and Ca:P=0.01-100), a source of fluoride ions, a gel for stabilization of calcium and phosphate ions, and a compound for prevention of nucleation, i.e. ethylene-diamine-tetramethylenphosphoric acid or its water soluble salts. The pH of preparation is between 5-9, preferably between 6.8-7.5, which mimics physiological conditions.

W. M. Jarvis and K. Y. Kim (U.S. Pat. No. 4,244,931: Dicalcium phosphate dehydrate with improved stability) discusses a preparation for polishing teeth containing dicalcium phosphate, and a sufficient amount of trimagnesiumphosphate and/or pyrophosphate which prevents spontaneous decomposition of dicalcium phosphate dehydrate.

H. Raaf, H. Harth and H. R. Wagner (U.S. Pat. No. 4,397,837: Process and composition for the remineralization and prevention of animal teeth including humans) discusses a preparation for remineralization and prevention of demineralization of dental enamel, containing two phases; one containing a water solution of calcium salts (50-35000 ppm and 0.005 wt. %-3.5 wt. %, respectively), and the other containing a water solution of phosphate (50-40000 ppm and 0.004 wt. %-4 wt. %, respectively) and water solution of fluoride (0.01 wt. %-5 wt. %). The preparation can also contain a polishing agent, astringent and preservatives.

A. G. Kolesnik, G. I. Kadnikova, L. V. Morozova and L. M. Boginskaya (U.S. Pat. No. 4,419,341: Drug for treatment of dental caries) describes a procedure to form a preparation for prevention of caries. A solution is prepared by dissolution of a mineral component and water soluble proteins from bone tissue by diluted mineral acid, and diluted by water. Lemon acid or lemon acid salt is added in such diluted solution as a stabilizer. The obtained solution is neutralized, evaporated, and then mixed with a pharmaceutical diluent in the ratio of 1:23.5-1:24.5.

J. J. Paran Jr. and N. Y. Sakkaab (U.S. Pat. No. 4,515,772: Oral compositions) discusses a preparation for protection of teeth in the form of a toothpaste containing 10-70% abrasives including metaphosphates, aluminum trioxide, polymerized resins and amorphous silica, 50-3500 ppm F ions, and at least 1.5% of P₂O₇⁴⁻ ions added in the form of dialkali metal and tetraalkali metal pyrophosphates and water. The preparation contains a maximum of 4% K₄P₂O₇ and the pH is between 6 and 10.

M. A. Rudy and V. F. Lisanti (U.S. Pat. No. 4,606,912: Method for making a clear, stable aqueous mouthwash solution and the solution made by that method for the enhancement of cells of the oral cavity and the remineralization of teeth) describes the preparation of a mouthwash for prevention of caries and reduction of unpleasant odor. The solution contains calcium chelates where minimally 50% of calcium ions is chelated. The solution is weakly alkaline.

F. J. Dany, H. Klassen, H. Prell and G. Kalteyer (U.S. Pat. No. 4,931,272: Toothpastes, cleaning agent for toothpastes based on dicalcium phosphate-dihydrate, and process for making such cleaning agent) discusses a procedure for preparation of a toothpaste containing dicalcium phosphate dihydrate as the main active component. The toothpaste contains more than 60% water per 100 g of active component.

M. J. Greenberg (U.S. Pat. No. 5,378,131: Chewing gum with dental health benefit employing calcium glycerophosphate) describes preparation of a chewing gum without fluorides that prevents development of dental caries and enhances dental hygiene, especially after meals containing fermentable carbohydrates. The chewing gum contains minimally 0.5 wt. % calcium glycerophosphate.

A. E. Winston and N. Usen (U.S. Pat. No. 5,603,922: Processes and compositions for the remineralization of teeth) describes a preparation for remineralization of teeth which contains two components: (1) 0.05-15% of one or more water soluble calcium salts and 0.001-2% of one or more water soluble divalent metals other than calcium; (2) 0.05-15% of one or more water soluble phosphate salts. After mixing together both components, a stable solution having pH between 4 and 7 is formed. During application and contact with teeth, remineralization occurs by diffusion of calcium and phosphate ions through the solution to the teeth surface, where hydroxyapatite is formed by the reaction between calcium and phosphate ions.

A. E. Winston and N. Usen (U.S. Pat. No. 5,614,175: Stable single-part compositions and the use of thereof for remineralization of lesions in teeth) describes a non-aqueous composition for remineralization of teeth and its application. The composition consists of 0.05-15% of one or more water soluble calcium salts, 0.05-15% of one or more water soluble phosphate salts, stabilizer, and up to 7% compounds for drying and coating. The pH of the composition is between 4.5 and 10.

A. E. Winston and N. Usen (U.S. Pat. No. 5,645,853: Chewing gum compositions and the use of thereof for remineralization of lesions in teeth) describes a chewing gum containing 0.01-15% of one or more water soluble calcium salts, 0.01-15% of one or more water soluble phosphate salts, 10-95% gum base and a layer for encapsulation. During chewing, both calcium and phosphate ions are released from the gum and together with saliva form a mixed solution of calcium and phosphate ions having a pH between 4 and 7. Phosphate and calcium ions from saliva deposit on the tooth surface where they react and induce remineralization by crystallization of calcium phosphate (hydroxyapatite).

L. C. Chow and S. Takagi (U.S. Pat. No. 5,695,729: Calcium phosphate hydroxyapatite precursor and methods for making and using the same) describes a procedure for preparation of a calcium phosphate composition usable in orthopedic and dental cements and remineralizers. The composition consists of tetracalcium phosphate prepared by a mixture of calcium and phosphorus in a ratio less than 1:2.

A. E. Winston and N. Usen (U.S. Pat. No. 5,817,296: Processes and compositions for the remineralization of teeth) describes a procedure for preparation of a stable non-aqueous dry composition which forms a water solution for remineralization of teeth after dissolution in water. The composition is prepared by dry mixing of one or more water soluble calcium salts (1-80%), one or more water soluble non-toxic salts of divalent metals other than calcium (0.1-20%), one or more water soluble phosphate salts, flavor (0.1-20%), sweetener (0.1-30%), one or more fluoride salts (0-10%) and surface active substances (about 5%). The pH of the water solution composition is between 4 and 7.

L. C. Chow, S. Takagi and G. L. Vogel (U.S. Pat. No. 5,833,954: Anti-carious chewing gums, candies, gels, toothpastes and dentifrices) describes a two-component preparation for remineralization of subsurface lesions and/or exposed dental tabulus in tooth. The cationic component contains one or more water soluble calcium salts, one or more water soluble non-toxic salts of divalent metals other than calcium and a pharmaceutically acceptable carrier. The anionic component contains one or more water soluble calcium salts, one or more water soluble fluoride salts and a pharmaceutically acceptable carrier. If the carrier of the cationic component is aqueous, then the carrier of anionic component is non-aqueous or hydrophobic. Similarly, if the carrier of the anionic component is aqueous, then the carrier of the cationic component is non-aqueous. Mixing of both components with water or saliva causes simultaneous release of calcium and phosphate ions and their mutual reaction on the tooth surface. If the contact of the remineralizing solution and tooth is long enough, calcium and phosphate ions diffuse through the tooth surface enabling remineralization of lesions and open dentinal tubules.

N. Usen and A. E. Winston (U.S. Pat. No. 5,895,641: Process and composition for remineralization and prevention of demineralization of dental enamel) describes a method for remineralization of lesions and open dentinal tubules in the subsurface layer of tooth which consists of: (1) preparation of a cationic component containing 0.05-15% of one or more water soluble calcium salts such as calcium chloride or nitrate; (2) preparation of an anionic component containing 0.05-15% of one or more water soluble phosphate salts and 0.01-5 water soluble fluoride salts; (3) a water solution having a pH between 4.5 and 10 is formed after mixing together anionic and cationic components where the solution contains free calcium ions released from calcium salts, free phosphate ions released from phosphate salts, and free fluoride ions released from fluoride salts. Application of the solution immediately after preparation causes the reaction of calcium, phosphate and fluoride ions on the surface of tooth. If the contact between the remineralizing solution and tooth is long enough, the calcium and phosphate ions diffuse through the surface of tooth which enables remineralization of lesions, dental plaque, open dentinal tubules, and exposed dentin.

A. E. Winston and N. Usen (U.S. Pat. No. 6,036,944: Process for remineralization of teeth) describes a method of remineralization of subsurface lesions and open dentinal tubules by preparation of components containing one or more water soluble calcium salts, one or more water soluble non-toxic salts of divalent metals different from calcium, one or more water soluble phosphate salts and mixing together the components so that the formed carbonateless solution has pH between 4.5 and 7. If the contact between the remineralizing solution and tooth is long enough, calcium and phosphate ions diffuse through the surface of tooth which enables remineralization of lesions dental plaque, open dentinal tubules, and exposed dentin.

Other approaches in remineralization utilize the properties of amorphous calcium phosphates (ACP) (M. S. Tung, U.S. Pat. No. 5,037,639; M. S. Tung, T. O'Farrell and D. W. Liu, J. Dent. Res. 72 (1993) 320). Among the different forms of calcium phosphates, ACP exhibits the maximum rate of formation, and maximum solubility, whereas ACP rapidly hydrolyzes into crystalline apatite ((E. D. Eanes in: Z. Amjad (Ed.), Calcium Phosphates in Biological and Industrial Systems, Kluwer Academic Pub., Boston, 1998, p. 21). High concentrations of calcium and phosphate ions from primary sources such as soluble salts rapidly precipitate ACP during their application as preparations for rinsing and nursing of teeth (M. S. Tung, M. Markovic and T. J. O'Farrell, J. Dent. Res. 73 (1994) 1903.; M. S. Tung, J. Dent. Res. 75 (1996) 56).

J. D. Termine, R. D. Peckauskas and A. S. Posner (Arch. Biochim. Biophys. 140 (1970) 318) established that when ACP is stabilized with pyrophosphate (P₂, O₇⁴⁻), the supersaturated solution is stable for a longer time. That is, spontaneous crystallization of the crystal forms of calcium phosphate is prevented.

Based on these findings, D. Skrtic, E. D. Eanes and J. M. Antonucci (in: C. G. Gebelein, C. E. Carraher, Jr. (Eds), Industrial Biotechnological Polymers, Technomic, Lancaster, Pa., 1995, p. 393) made discs formed of ACP stabilized with pyrophosphate incorporated in metaacrylic resins. When such discs are immersed in buffered salted solution, they release calcium and phosphate ions in concentrations sufficient to form a stable solution supersaturated with respect to hydroxyapatite.

Soluble ACP may be applied as an additive in chewing gum. Calcium, phosphate, fluoride, bicarbonate and hydroxyl ions needed for remineralization and regulation of pH in the dental cavity are released during chewing (M. S. Tung and F. C. Eichmiller, J Clin. Dent. 10 (1991) 1).

One preparation for remineralization of teeth based on ACP is the sugarless chewing gum Recaldent™ in which the source of calcium and phosphate ions is ACP stabilized by casein, i.e. part of protein from cow milk. Recaldent™ was developed and patented by the School of Dental Science, University of Melbourne, Australia and exclusively licensed by Bonlac Foods.

L. Dent, E. P. Hertzenberg and H. S. Sherry (U.S. Pat. No. 4,349,533: Toothpaste containing pH-adjusted zeolite) describes the method of adjusting the pH value of oral compositions in the range pH=5.5 to pH=6 using zeolite NaHA, CaHA, MgHA, NaHX, CaHX, ZnHX, MgHX and their mixtures obtained by ion exchange and acid treatment (modification) of sodium forms of zeolite.

Weststrate et al. (U.S. Pat. No. 4,460,565) developed a preparation for remineralization containing 1000-15000 ppm of F ions, depending on use, applied in the form of alkaline fluorides, earth alkaline fluorides, ammonium fluoride and alkaline fluorophosphates, 0.1-5 wt. % soluble cyclic alkaline phosphates, 0.05-5 wt. % of calcium containing substances, e.g. calcium citrate, calcium tartarate, calcium adipate, calcium apophyllite and calcium zeolite, and soluble linear phosphates so that the atomic ratio Ca:P is about 1.66:1. Along with an entire series of soluble calcium salts (citrates, tartarates, adipates, apophyllates), the calcium form of zeolites may also be used as a source of calcium ions. In example 2, calcium tartrate used as the only source of calcium ions. In example 3, the main source of calcium is calcium citrate and calcium zeolite, respectively. The influence of zeolites as source of calcium on the effect of remineralization was not tested.

R. S. Schreiber and J. R. Principe (U.S. Pat. No. 4,187,287: Warm Two Tone Flavored Dentifrice) describes application of dehydrated forms of zeolite 3A, 4A and 5A as well as zeolite X as components to increase the temperature of oral compositions, and hence, enhance the effect of flavor components. At the same time, the abrasive and polishing effects of zeolite may reduce the amounts of other abrasive and polishing agents. However, while the abrasive and polishing effects of zeolite are doubtless, the effect of “warming up” is time-limited because of the reversible character of adsorption and desorption of zeolitic water. The thermal effect can be effective just at the time of mixing of zeolite with other components of the oral composition, but this effect completely disappears after a certain time passes between the preparation and application of the oral composition.

J. E. Barry, et al. (U.S. Pat. No. 6,123,925) disclose an antibiotic toothpaste with an antibiotic inorganic metal containing composition present in an amount effective to impart substantial antimicrobial activity within the normal time for brushing teeth. Zeolites, mainly zeolite A, were used as carriers of the antibiotic metal ions such as silver, gold, copper and zinc. In the antibiotic zeolite particles used in the invention, exchangeable ions present in zeolite such as sodium, calcium, potassium and iron are preferably partially replaced with ammonium and antibiotic metal ions.

Most of the methods and corresponding preparations for dental hygiene presented hereinabove use water soluble calcium salts as a source of calcium ions and water soluble phosphate salts as a source of phosphate ions in the process of remineralization of teeth. However, implementing water soluble and/or partially soluble calcium and phosphate salts as a source of calcium and phosphate ions in the agents for the remineralization induces difficulties connected with control of concentration of calcium and phosphate ions. If the concentrations of the ions are too low, one cannot reach the required level of remineralization. On the other hand, too high concentrations of the ions can cause crystallization of apatites with defective crystalline structure and/or unwanted crystal agglomerates on teeth surfaces. The problem is a permanent change of the concentration of calcium and phosphate ions in the solution during the process of remineralization. Furthermore, water soluble and/or partially soluble calcium and phosphate salts are a source of various negative anions (chlorides, nitrates, bicarbonates etc.) which may have negative impact on the crystallization of hydroxyapatite, and thus, on the stability and solubility of dental enamel. Additionally, due to relatively low concentrations of phosphates and further reduction during remineralization, the reduction of pH (i.e. increased acidity) in the dental cavity causes slowing down of the remineralization process or even an increase in rate of demineralization.

Since control of acidity (pH) in the dental cavity is one of the most significant factors for the control of stability of the mineral portion of enamel and dentine, and control of the development of dental caries (M. E. Jensen, Cariology 43 (1999) 615), in the above methods and corresponding preparations, the pH is controlled by methods such as addition of bicarbonate and/or urea. Although bicarbonates present in preparations for dental hygiene reduce acidity and increase stability of the mineral part of teeth, the presence of carbonates can also have negative impact on the stability of dental enamel and dentin. Specifically, OH⁻ ions in hydroxyapatite can be replaced with carbonate ions from the solution, and form carbonated apatites which in an acid environment are more soluble than hydroxyapatite. Urea, although a neutral substance, in an acid environment causes hydrolysis of ammonium and carbon dioxide supported by the activities of cariogenic bacteria. The formed ammonia neutralizes the acid and reduces acidity in the mouth cavity. However, more recent studies comparing the use of commercial chewing gums without urea and chewing gums with urea, have proven that the presence of urea has no significant effect on dental cavity pH (D. Birkhed, J. Dent Res. 68 (Special Issue) (1989): Abstract 1027.;T Imfeld, Telemetric Evaluation Discourse the Plaque pH Neutralize Potential of two Chewing Gums Provided by Fertin A/S., Intermural Paper, Dental Institute of Zurich, September 1996). Furthermore, it is known that ammonium reduces the lifetime of cells and induces the growth of human gingival fibroblasts in vivo (K. Helgeland, Scand. J Res. 89 (1981) 400). Ammonium negatively affects the emission of collagen by virtue of cells and can provoke periodontal inflammation and tissue breakdown (K. Helgeland, Scand. J Dent. Res. 92 (1984) 419; K. Helgeland, Scand. J Dent. Res. 93 (1981) 39). Finally, carbon dioxide formed as a product of urea hydrolysis can form carbonate which can replace OH⁻ ions in hydroxyapatite, and can form carbonated apatites which are much more soluble than hydroxyapatite in an acidic environment. Hence, it follows that application of urea is not only questionable regarding any neutralization of acidity, but it can cause health problems.

Although zeolites as ingredients in oral compositions appeared in several patents (e.g., L. Dent, et al. U.S. Pat. No. 4,349,533; Schreiber and Principe U.S. Pat. No. 4,187,287; Weststrate, et al. U.S. Pat. No. 4,460,565; Barry, et al. U.S. Pat. No. 6,123,925), the calcium form of zeolite is mentioned as a possible source of calcium ions in only two patents, U.S. Pat. No. 4,349,533 and U.S. Pat. No. 4,460,565 neither of which teach the present invention.

In U.S. Pat. No. 4,349,533 (L. Dent et al.), the method of pH control by using the Me, H-forms of zeolite A was described. However, because the pH achieved by using zeolite is too low for the effective prevention of demineralization processes, zeolites modified by acid treatment are unstable and tend to transform into amorphous aluminosilicates, especially zeolite A, during both the acid treatment and the time passed from preparation of the oral composition to its application.

In U.S. Pat. No. 4,460,565 to Weststrate, et al., taking into account the small percentage of calcium supplied by the calcium form of zeolite (0.5 wt. %), the maximum amount of calcium ions arising from the calcium form of zeolite represents 0.05 wt. % of the total formulation. This represents only a minute amount of the total calcium ions in the formulation. The main source of Ca²⁺ ions is calcium citrate, and thus the calcium supplied by the calcium form of zeolite can only be considered as an ‘auxiliary’ or trace source of calcium. Since the calcium ions from citrate are ‘free’ and the calcium ions from zeolite must be ‘released’ from the zeolite framework before they can be available to react with phosphate ions, the calcium ions arising from calcium zeolite cannot considerably affect the remineralization process. In addition, although the pH of the preparations described in this patent are not specified, at a constant atomic ratio of Ca:P=1.66:1, such a small amount of zeolite cannot considerably participate in the regulation and adjustment of pH values, which due to the presence of citric or phosphoric acid is usually less than 7.

Finally, a preferably partial replacement of zeolite host cations with ammonium and antibiotic metal ions (J. E. Barry et al. (U.S. Pat. No. 6,123,925) means that even when originally used zeolites contain cations that are different from the antibiotic ions, these original host cations, including calcium cations (although use of “mainly zeolite A” makes uncertain the presence of the host cations other than sodium cations), are exchanged with the antibiotic ions. Hence, it is obvious that the toothpaste does not contain a calcium form of zeolite, and thus, zeolite is not used as the source of calcium, but exclusively as an antibacterial agent, as declared in the patent. Zeolite is not used for the regulation and adjustment of pH, because it is regulated in a most simple and primitive way by the addition of NaOH.

Taking into consideration the above mentioned difficulties in controlling the pH via the use of bicarbonate, urea and acid modified zeolite and the concentrations of calcium and phosphate ions used previously for teeth remineralization, there remains a need for effective oral compositions for simultaneous stabilization, i.e., decrease of demineralization, recalcification and remineralization of tooth enamel and dentin and efficient protection of teeth against caries.

The present inventors have discovered that, most of the above-mentioned problems, i.e., poor control of calcium and phosphate ion concentrations and related problems with insufficient levels of remineralization or the uncontrolled rate of formation of hydroxyapatite having defective crystal structure; presence of anions which can destabilize the crystal structure of hydroxyapatite; use of urea and bicarbonates in controlling pH, etc. can be prevented by use of zeolites as sources of calcium ions.

### SUMMARY OF THE INVENTION

In various embodiments, the present invention is directed to: (a) an oral composition for stabilization, recalcification and remineralization of dental enamel and dentin based on the controlled release of calcium ions from the calcium form of zeolite in the presence of water soluble phosphate salts with or without dental matrix proteins; and (b) a process for use of same.

The calcium ion source preferably comprises a calcium form of zeolite of type I, II and III. The calcium form of zeolite of type I, II and III is between about 0.1-10 wt. % of the composition.

In one aspect, the release of calcium ions from the calcium form of zeolite is controlled release.

The phosphate ion source preferably comprises a water soluble sodium phosphate such as Na₃PO₄, Na₂HPO₄ and NaH₂PO₄, and preferably, Na₂HP0₄. The phosphate ions are between about 0.00132-1.586 wt. % of the composition.

Hence, the molar ratio of calcium to phosphate (Ca/P) is from 0 to about 170.5.

In another embodiment, a controlled reaction between the released calcium ions and dissolved phosphate ions occurs on a tooth surface.

In one aspect, the enamel matrix proteins comprise between 1.32×10⁴−0.1 wt. % of the composition. The enamel matrix proteins increase remineralization rates.

In another embodiment, the oral composition for stabilization, recalcification and remineralization of dental enamel and dentin comprises: 0.1-10 wt. % of the calcium form of zeolite; 0.00132-2 wt. % of phosphate ions; 1.32×10⁻⁴−0.1 wt. % of enamel matrix proteins; and one or more additional ingredients such as, abrasives, thickening agents, binding agents, surface acting agents, sweeteners, corigenses of taste, solvents, and mixtures thereof. In another embodiment, the oral composition contains between 0.01-0.025 wt. % of an milfoil extract and/or oil.

In another embodiment, the invention is to an oral composition for stabilization, recalcification and remineralization of dental enamel and dentin based on the controlled release of calcium ions from the calcium form of zeolite in the presence of water soluble phosphate salts with or without dental matrix proteins where the pH of the oral composition ranges between about 4.88 to about 11.82, preferably 8.12 to about 8.50. The pH of the oral composition is unaffected by dilution. In one embodiment, where the pH of the oral composition between about 7.5 to about 11.9, hydroxyapatite is stabilized and formed on teeth during remineralization. The enamel matrix proteins increase stability of hydroxyapatite formed on teeth during remineralization about 30-50%.

In one embodiment, the oral composition is a toothpaste. In other embodiments, the oral composition is a chewing gum, bonbons, candy, confectionaries, mouth rinses, films, and lozenges.

In another embodiment, the invention is directed to an oral composition for stabilization, recalcification and remineralization of dental enamel and dentin where the calcium ion source, phosphate ion source, and enamel matrix proteins are physically separated, such as located in different layers or via micro encapsulation.

In another embodiment, the invention is directed to a process for stabilization, recalcification and remineralization of dental enamel and dentin comprising: application of a composition containing a controlled release of calcium ions from the calcium form of zeolite in the presence of water soluble phosphate salts with or without dental matrix proteins wherein the calcium ion source preferably comprises a calcium form of zeolite of type I, II and III, wherein the calcium form of zeolite of type I, II and III is between about 0.1-10 wt. % of the composition, wherein release of calcium ions from the calcium form of zeolite is controlled release, wherein the phosphate ion source preferably comprises a water soluble sodium phosphate such as Na₃PO₄, Na₂HPO₄ and NaH₂PO₄, and preferably, Na₂HP0₄, wherein the phosphate ions are between about 0.00132-1.586 wt. % of the composition, wherein the molar ratio of calcium to phosphate (Ca/P) is from 0 to about 170.5, wherein the enamel matrix proteins comprise between 1.32×10⁻⁴−0.1 wt. % of the composition, and wherein the enamel matrix proteins increase remineralization rates.

In another embodiment, the invention is directed to a process for stabilization, recalcification and remineralization of dental enamel and dentin comprising: application of a composition containing a controlled release of calcium ions from the calcium form of zeolite in the presence of water soluble phosphate salts with or without dental matrix proteins and the controlled reaction between the released calcium ions and dissolved phosphate ions occurs on a tooth surface.

In another embodiment, the invention is directed to a process for stabilization, recalcification and remineralization of dental enamel and dentin comprising: application of: 0.1-10 wt. % of the calcium form of zeolite; 0.00132-2 wt. % of phosphate ions; 1.32×10⁻⁴−0.1 wt. % of enamel matrix proteins; and one or more additional ingredients such as, abrasives, thickening agents, binding agents, surface acting agents, sweeteners, corigenses of taste, solvents, and mixtures thereof. In another embodiment, the process involves application of the oral composition which may also contain between 0.01-0.025 wt. % of an milfoil extract and/or oil.

In another embodiment, the invention is to a process for stabilization, recalcification and remineralization of dental enamel and dentin based on the controlled release of calcium ions from the calcium form of zeolite in the presence of water soluble phosphate salts with or without dental matrix proteins where the pH of the oral composition ranges between about 4.88 to about 11.82, preferably 8.12 to about 8.50. The pH of the oral composition is unaffected by dilution. The process involves stabilization and formation of hydroxyapatite on teeth during remineralization. The enamel matrix proteins increase stability of hydroxyapatite formed on teeth during remineralization about 30-50%. The process may be directed to application of an oral composition which is a toothpaste, chewing gum, bonbons, candy, mouth rinse, film, and lozenge.

In another embodiment, the invention is directed to a process for stabilization, recalcification and remineralization of dental enamel and dentin by application of a composiiton where the calcium ion source, phosphate ion source, and enamel matrix proteins are physically separated, such as located in different layers or via micro encapsulation. The process is useful for specific requirements of dental enamel of pregnant women, older persons and children, in cases of osteoporosis as well as prophylaxis and treatment of gingivitis.

In another aspect of the invention, the oral composition for stabilization, recalcification and remineralization of dental enamel and dentin described above may be in the form of a kit containing directions for use.

### BRIEF DESCRIPTION OF THE DRAWINGS

The present invention will be better understood in view of the following non-limiting figures, wherein:

FIG. 1 provides a graphical illustration of the dependence of pH of the oral composition on the logarithm, log C_{P}, of the concentrations of PO₄³⁻, HPO₄²⁻, and H₂PO₄⁻ ions.

FIG. 2 provides a graphical illustration of the concentration of calcium ions in the solution after the demineralization of untreated teeth and teeth treated with water suspension of zeolite(s).

FIG. 3 provides a graphical illustration of the effect of the amount of hydroxyapatite deposited on the tooth on the time of remineralization.

FIG. 4 provides a graphical illustration of the influence of time, t, on the treatment of teeth with the oral composition for the stabilization, recalcification and remineralization of dental enamel and dentin, before demineralization, on the increase of average percentage, Sav, of the teeth stability.

### DETAILED DESCRIPTION OF THE INVENTION

In various embodiments, the present invention involves employing oral compositions for the stabilization, recalcification and remineralization of dental enamel and dentin and protection of teeth against tooth caries. The invention relates to the use of the calcium form of zeolite, water-soluble phosphate salts and matrix proteins of teeth for pH adjustment in the mouth and simultaneous building of calcium and phosphate ions from the oral solution into tooth enamel and dentin in the presence of tooth matrix proteins to stabilize the crystal structure of calcium hydroxyapatite in tooth enamel and dentin.

As used herein, the term “dental matrix protein” is defined as structural and/or adhesive and include those of enamel. Examples of the matrix proteins of the invention are collagen, elastin, ameloblastin, sheathlin, However, the term may also include amelogenins, praline-rich non-amelogenins, tuftelin, tuft proteins, serum proteins and other proteins. The terms “matrix proteins”, “dental matrix proteins”, “enamel matrix proteins” and “matrix proteins of the teeth” are used interchangeably throughout the specification.

Dentin is defined herein as the substance between the enamel or cementum and the pulp chamber. It is secreted by the odontoblasts of the dental pulp. An odontoblast is a cell involved in dentinogenesis, which is the creation of dentin, the substance under tooth enamel. The odontoblasts secrete dentin throughout life, which may be an attempt to compensate for natural wearing down of the enamel. These cells are responsible for producing the calcified dental matrix. The dental matrix protein is one of the dental noncollagenous matrix proteins that has been implicated in regulation of mineralization.

Since the calcium ions are bound in the microcrystalline particles of inorganic carriers (zeolite), they are not active until “free” from the zeolite crystal framework. Hence it follows, that although the concentrations of calcium and phosphate ions in the oral composition are favorable (or more than favorable) for total remineralization, the speed of remineralization is controlled by the rates of release of calcium ions from the zeolite microcrystals during the exchange of calcium ions from the zeolite with other ions from the solution. The calcium ions released from zeolite microcrystals are incorporated, together with equivalent quantity of phosphate ions into the dental enamel and dentin, and remineralize them. If the composition contains dental matrix proteins, the same improves the above mentioned processes by its action as the precursor for the transformation of the “unripe” calcium hydroxyapatite into the “ripe” calcium hydroxyapatite. “Ripe” calcium hydroxyapatite is more stable than “unripe” calcium hydroxyapatite. The components of calcium hydroxyapatite; calcium ions, phosphate ions and dental matrix protein integrally construct the dental enamel and/or dentin by a self-organized process (restitutio ad integrum).

In one aspect, the invention can be in the form of a toothpaste. In other aspects the oral composition may be in the form of chewing gum, gel, mouth rinse, candy, lozenge, film, transbuccal patch and other preparations which can be kept in the mouth cavity.

In various embodiments of the oral composition, the extract and/or the oil of the medicinal herb milfoil may be present in the amounts from about 0.01 to about 0.025 wt %, as a mild anti-inflammatory agent.

Although the essential role of the calcium form of zeolite in the oral composition for the stabilization, recalcification and remineralization of dental enamel (OKSRCD) based on this invention is supplying active calcium ions, the calcium form of zeolite has also another crucial role in the control and maintenance of optimal pH for the stabilization and remineralization of dental enamel and dentin. Table 1 shows the pH values of the liquid phases of the suspensions containing 1 g of the calcium form of zeolite (types I, II and III) in 10, 100 and 1000 ml of demineralized water.

TABLE 1
Influence of the type of calcium form of zeolite and
the volume of water in which 1 g of zeolite is suspended
on pH of the liquid phase of the suspension.

| | pH | | |
|---|---|---|---|
| | Concentration of suspension | | |
| Type of zeolite | 1 g/10 ml | 1 g/100 ml | 1 g/1000 ml |
| I | 9.05-9.20 | 8.00-8.10 | 7.00-7.01 |
| II | 8.10-8.20 | ≈7.50 | 6.00-6.40 |
| III | 7.50-7.60 | 7.01-7.05 | 4.92-5-03 |

The data in Table 1 shows that in all cases the starting pH of the demineralized water, i.e., 4.8 increased after addition of the calcium form of zeolite. For a constant concentration of suspension, expressed as the volume of water in which 1 g of zeolite is suspended, pH decreases in the sequence: pH(type I)=7-9.2>pH(type II)=6-8.2>pH(type III)=4.92-7.6. Hence, with the increase of the molar ratio Si/Al in the framework structure of zeolite, i.e., Si/Al(type D<Si/Al(type II)<Si/Al(type III). As shown in table 1, for a given type of zeolite, pH depends on the concentration of suspension, so that using calcium form of different types of zeolites (having Si/Al in the range from 1 to 10) in the suspensions containing 1 g of zeolite in 10, 100 and 1000 ml of water, the suspensions having pH in the range from about 5 to 9 can be obtained.

With respect to wide use of soluble phosphate salts for regulation of pH in the biological systems (J. Gabelberger, W. Liebl and K. H. Schleifer, Appl. Microbiol. Biotechnol.40 (1993) 44.;K. Uchida and S. Kawasaki, J. Biol. Chem. 269 (1994) 2405; Y. Kawata and K. Hamaguchi, Protein Sci. 4 (1995) 416; F. Ruizteran and J. D. Owens, Lett. Appl. Microbiol. 22 (1996) 30; K. Brogden and C. Clarke, Infect. Immun. 65 (1997) 957; H. Chen and M. R. Juchau, Drug Metabolism Disposition 26 (1998) 222.; P.E. Jorgensen, L. Eskildsen and E. Nexo, Scand. J. Clin. Lab. Invest. 59 (1999) 191; O. Castejon and P. Sims, Biocell. 23 (2000) 187; Y. Bai and Z. L. Nikolov, Biotechnol Prog. 17 (2001) 168; C. K. Chang, V. Simplaceanu and C. Ho, Biochemistry 41 (2002) 5644) including the methods and preparations used in the dental hygiene (K. Tomlinson and E J. Duuf, U.S. Pat. No. 4,048,300; D. N. DiGiulio and R J. Grabenstetter, U.S. Pat. No. 4,080,440; R J. Grabenstetter and J. A. Gray, U.S. Pat. No. 4,083,955; M. C. S. Gaffar and A. Gaffar, U.S. Pat. No. 4,177,258; H. Raaf, H. Harth and H. R. Wagner, U.S. Pat. No. 4,397,837; A. E. Winston and N. Usen, U.S. Pat. No. 5,603,922; A. E. Winston and N. Usen, U.S. Pat. No. 5,614,175; A. E. Winston and N. Usen, U.S. Pat. No. 5,645,853; A. E. Winston and N. Usen, U.S. Pat. No. 5,817,296; L. C. Chow, S. Takagi and G. L. Vogel, U.S. Pat. No. 5,833,954; N. Usen and A. E. Winston, U.S. Pat. No. 5,895,641; A. E. Winston and N. Usen, U.S. Pat. No. 6,036,944), each of which is incorporated herein in its entirety; soluble salts in various anionic phosphate forms (PO₄³⁻, HPO₄²⁻ and H₂PO₄⁻) can be used for regulation of pH in the mouth cavity, even more because the phosphate ion is one of the essential ingredients of natural apatites—the mineral part of dental enamel and dentin. Due to differences in the stability of different phosphate anions (stability increases in the sequence: H₃PO₄<H₂PO₄⁻<HPO₄²⁻<PO₄³⁻), it can be expected that the pH of the solution depends considerably on the type and concentration of dissolved (Na,H)-phosphate. Such an expectation is justified by the data presented in Table 2. All solutions of Na₃PO₄ and Na₂HPO₄ are alkaline (pH>7); in both the cases pH increases with increasing salt concentration. However, as expected, pH values of Na₃PO₄ solutions (9.27-11.8) are higher than pH values of the corresponding Na₂HPO₄ solutions (8.05-9.6). In contrast to solutions of Na₃PO₄ and Na₂HPO₄, the solutions of NaH₂PO₄ are acidic for all examined concentrations (pH<7); opposite to the solutions of Na₃P04 and Na₂HPO₄, pH of NaH₂PO₄ solutions decreases with increasing concentration.

TABLE 2
Influence of the type of phosphate salt and concentration
of its water solution on pH of the solution.

| | pH | | |
|---|---|---|---|
| | Conc of solution | | |
| | 1.67 × 10⁻³ | 1.67 × 10⁻² | 1.67 × 10⁻¹ |
| Type of phosphate | mol dm⁻³ | mol dm⁻³ | mol dm⁻³ |
| Na₃PO₄ | 9.27 | 11.75 | 11.80 |
| Na₂HPO₄ | 8.05 | 9.10 | 9.60 |
| NaH₂PO₄ | 3.30 | 3.14 | 2.90 |

Since processes of demineralization, remineralization and stabilization of dental enamel and dentin largely depend on the pH of liquid in the dental cavity, and thus, also on the pH of the oral composition, it is extremely important to know how pH of the mentioned oral composition depends on the type and content of the calcium form of zeolite as well as on the concentration of different sodium phosphates (Na₃PO₄, Na₂HPO₄ and NaH₂PO₄), phosphate salts in their mixtures. It is quite certain that the pH established by a combination of a calcium form of zeolite and soluble phosphate salts is different than the pH of both zeolite and phosphate alone. Hence, the influence of the mixtures of calcium form of zeolite(s) and different phosphates under different conditions (0.1-10 wt. % of zeolite, 0-1.586 wt. % of phosphate, molar ratio Ca/P=0.16-∞, where types I, II and III of a calcium form of zeolite were used as the sources of calcium ions and Na₃PO₄, Na₂HPO₄ and NaH₂PO₄ were used as the source of phosphate ions). The pH of the mixtures was determined and listed in Table 3. The concentration of dental matrix proteins in the oral composition is too small to have any significant effect on the pH of the oral composition, and is not considered. Similarly, the weight ratios of matrix proteins are not mentioned in the table, because they have no influence on the pH.

TABLE 3
Influence of the type of calcium form of zeolite (CaZ), weight percent of calcium
form of zeolite (wt. % CaZ), chemical forms of phosphate ions, weight percent of
phosphate ions (wt. % of phosphate) and molecular concentrations of phosphate ions
and molar ratio Ca/P on the equilibrium pH value of the oral composition for stabilization,
recalcification and remineralization of dental enamel and dentin.

| | type | weight | form of | wt. % of | molar concentration | molar | equilibrium |
|---|---|---|---|---|---|---|---|
| No. | CaZ | % CaZ | phosphate | phosphate | of phosphate mol dm−³ | ratio Ca/P | state of pH |
| 1. | I | 0.1 | — | 0 | 0 | ∞ | 7.50 |
| 2. | I | 0.1 | PO₄³⁻ | 0.1586 | 0.0167 | 0.16 | 11.70 |
| 3. | I | 0.1 | PO₄³⁻ | 0.01586 | 0.00167 | 1.6 | 9.80 |
| 4. | I | 0.5 | PO₄³⁻ | 0.1586 | 0.0167 | 0.8 | 11.68 |
| 5. | I | 1.0 | — | 0 | 0 | ∞ | 9.07 |
| 6. | I | 1.0 | PO₄³⁻ | 0.1586 | 0.0167 | 1.6 | 11.72 |
| 7. | I | 1.0 | PO₄³⁻ | 0.075 | 0.0079 | 3.38 | 11.31 |
| 8. | I | 1.0 | PO₄³⁻ | 0.0076 | 0.0008 | 33.4 | 8.40 |
| 9. | I | 1.0 | PO₄³⁻ | 0.00715 | 0.00075 | 35.6 | 8.30 |
| 10. | I | 1.0 | PO₄³⁻ | 0.00355 | 0.00037 | 71.4 | 7.96 |
| 11. | I | 1.0 | PO₄³⁻ | 0.00143 | 0.00015 | 178 | 8.15 |
| 12. | I | 5.0 | PO₄³⁻ | 1.586 | 0.167 | 0.8 | 11.72 |
| 13. | I | 10.0 | — | 0 | 0 | ∞ | 9.18 |
| 14. | I | 10.0 | PO₄³⁻ | 1.586 | 0.167 | 1.6 | 11.74 |
| 15. | I | 0.1 | HPO₄²⁻ | 0.1586 | 0.0167 | 0.16 | 8.16 |
| 16. | I | 0.1 | HPO₄²⁻ | 0.01586 | 0.00167 | 1.6 | 8.20 |
| 17. | I | 0.5 | HPO₄²⁻ | 0.1586 | 0.0167 | 0.8 | 8.00 |
| 18. | I | 1.0 | HPO₄²⁻ | 1.586 | 0.167 | 0.16 | 8.05 |
| 19. | I | 1.0 | HPO₄²⁻ | 0.632 | 0.0665 | 0.4 | 8.10 |
| 20. | I | 1.0 | HPO₄²⁻ | 0.316 | 0.0333 | 0.8 | 7.96 |
| 21. | I | 1.0 | HPO₄²⁻ | 0.1586 | 0.0167 | 1.6 | 8.10 |
| 22. | I | 1.0 | HPO₄²⁻ | 0.0534 | 0.00562 | 4.75 | 7.92 |
| 23. | I | 1.0 | HPO₄²⁻ | 0.027 | 0.00284 | 9.4 | 7.85 |
| 24. | I | 1.0 | HPO₄²⁻ | 0.00726 | 0.000764 | 34.95 | 7.81 |
| 25. | I | 1.0 | HPO₄²⁻ | 0.00395 | 0.000416 | 64.18 | 7.80 |
| 26. | I | 1.0 | HPO₄²⁻ | 0.00132 | 0.000139 | 182 | 7.85 |
| 27. | I | 5.0 | HPO₄²⁻ | 1.586 | 0.167 | 0.8 | 8.16 |
| 28. | I | 10.0 | HPO₄²⁻ | 1.586 | 0.167 | 1.6 | 8.15 |
| 29. | I | 0.1 | H₂PO₄⁻ | 0.1586 | 0.0167 | 0.16 | 6.93 |
| 30. | I | 0.1 | H₂PO₄⁻ | 0.01586 | 0.00167 | 1.6 | 7.20 |
| 31. | I | 0.5 | H₂PO₄⁻ | 0.1586 | 0.0167 | 0.8 | 6.82 |
| 32. | I | 1.0 | H₂PO₄⁻ | 0.158 | 0.0167 | 1.6 | 6.78 |
| 33. | I | 1.0 | H₂PO₄⁻ | 0.0071 | 0.00075 | 35.7 | 7.60 |
| 34. | I | 1.0 | H₂PO₄⁻ | 0.00364 | 0.00038 | 69.5 | 7.90 |
| 35. | I | 1.0 | H₂PO₄⁻ | 0.00194 | 0.000204 | 130.5 | 7.68 |
| 36. | I | 5.0 | H₂PO₄⁻ | 1.586 | 0.167 | 0.8 | 6.56 |
| 37. | I | 10.0 | H₂PO₄⁻ | 1.586 | 0.167 | 1.6 | 6.65 |
| 38. | II | 0.1 | — | 0 | 0 | ∞ | 6.10 |
| 39. | II | 0.1 | PO₄³⁻ | 0.001586 | 0.000167 | 12.8 | 8.15 |
| 40. | II | 0.1 | PO₄³⁻ | 0.01586 | 0.00167 | 1.28 | 9.62 |
| 41. | II | 0.1 | PO₄³⁻ | 0.1586 | 0.0167 | 0.128 | 11.76 |
| 42. | II | 1.0 | — | 0 | 0 | ∞ | 6.35 |
| 43. | II | 1.0 | PO₄³⁻ | 0.001586 | 0.000167 | 128 | 8.17 |
| 44. | II | 1.0 | PO₄³⁻ | 0.01586 | 0.00167 | 12.8 | 9.76 |
| 45. | II | 1.0 | PO₄³⁻ | 0.1586 | 0.0167 | 1.28 | 11.78 |
| 46. | II | 10.0 | — | 0 | 0 | ∞ | 7.90 |
| 47. | II | 10.0 | PO₄³⁻ | 0.01586 | 0.00167 | 128 | 9.58 |
| 48. | II | 10.0 | PO₄³⁻ | 0.1586 | 0.0167 | 12.8 | 11.67 |
| 49. | II | 10.0 | PO₄³⁻ | 1.586 | 0.167 | 1.28 | 11.82 |
| 50. | II | 0.1 | HPO₄²— | 0.001586 | 0.000167 | 12.8 | 7.85 |
| 51. | II | 0.1 | HPO₄²— | 0.01586 | 0.00167 | 1.28 | 7.96 |
| 52. | II | 0.1 | HPO₄²— | 0.1586 | 0.0167 | 0.128 | 8.12 |
| 53. | II | 1.0 | HPO₄²— | 0.001586 | 0.000167 | 128 | 7.78 |
| 54. | II | 1.0 | HPO₄²— | 0.01586 | 0.00167 | 12.8 | 7.91 |
| 55. | II | 1.0 | HPO₄²— | 0.1586 | 0.0167 | 1.28 | 8.05 |
| 56. | II | 10.0 | HPO₄²— | 0.01586 | 0.00167 | 128 | 8.00 |
| 57. | II | 10.0 | HPO₄²— | 0.1586 | 0.0167 | 12.8 | 8.06 |
| 58. | II | 10.0 | HPO₄²— | 1.586 | 0.167 | 1.28 | 8.16 |
| 59. | II | 0.1 | H₂PO₄⁻ | 0.001586 | 0.000167 | 12.8 | 7.78 |
| 60. | II | 0.1 | H₂PO₄⁻ | 0.01586 | 0.00167 | 1.28 | 7.44 |
| 61. | II | 0.1 | H₂PO₄⁻ | 0.1586 | 0.0167 | 0.128 | 6.60 |
| 62. | II | 1.0 | H₂PO₄⁻ | 0.001586 | 0.000167 | 128 | 7.80 |
| 63. | II | 1.0 | H₂PO₄⁻ | 0.01586 | 0.00167 | 12.8 | 7.54 |
| 64. | II | 1.0 | H₂PO₄⁻ | 0.1586 | 0.0167 | 1.28 | 6.66 |
| 65. | II | 10.0 | H₂PO₄⁻ | 0.01586 | 0.00167 | 128 | 7.65 |
| 66. | II | 10.0 | H₂PO₄⁻ | 0.1586 | 0.0167 | 12.8 | 6.76 |
| 67. | II | 10.0 | H₂PO₄⁻ | 1.586 | 0.167 | 1.28 | 6.28 |
| 68. | III | 0.1 | — | 0 | 0 | ∞ | 4.88 |
| 69. | III | 0.1 | PO₄³⁻ | 0.001586 | 0.000167 | 9.6 | 8.07 |
| 70. | III | 0.1 | PO₄³⁻ | 0.01586 | 0.00167 | 0.96 | 9.46 |
| 71. | III | 0.1 | PO₄³⁻ | 0.1586 | 0.0167 | 0.096 | 11.56 |
| 72. | III | 1.0 | — | 0 | 0 | ∞ | 5.50 |
| 74. | III | 1.0 | PO₄³⁻ | 0.001586 | 0.000167 | 96 | 8.08 |
| 75. | III | 1.0 | PO₄³⁻ | 0.01586 | 0.00167 | 9.6 | 9.52 |
| 76. | III | 1.0 | PO₄³⁻ | 0.1586 | 0.0167 | 0.96 | 11.63 |
| 77. | III | 10.0 | — | 0 | 0 | ∞ | 6.85 |
| 78. | III | 10.0 | PO₄³⁻ | 0.01586 | 0.00167 | 0.96 | 9.61 |
| 79. | III | 10.0 | PO₄³⁻ | 1.586 | 0.167 | 0.96 | 11.73 |
| 80. | III | 0.1 | PO₄³⁻ | 0.001586 | 0.000167 | 9.6 | 7.65 |
| 81. | Ill | 0.1 | HPO₄²⁻ | 0.01586 | 0.00167 | 0.96 | 7.80 |
| 82. | III | 0.1 | HPO₄²⁻ | 0.1586 | 0.0167 | 0.096 | 8.04 |
| 83. | III | 1.0 | HPO₄²⁻ | 0.001586 | 0.000167 | 96 | 7.68 |
| 84. | III | 1.0 | HPO₄²⁻ | 0.01586 | 0.00167 | 9.6 | 7.84 |
| 85. | III | 1.0 | HPO₄²⁻ | 0.1586 | 0.0167 | 0.96 | 7.96 |
| 86. | III | 10.0 | HPO₄²⁻ | 0.01586 | 0.00167 | 96 | 8.01 |
| 87. | III | 10.0 | HPO₄²⁻ | 0.1586 | 0.0167 | 9.6 | 8.06 |
| 88. | III | 10.0 | HPO₄²⁻ | 1.586 | 0.167 | 0.96 | 8.19 |
| 89. | III | 0.1 | H₂PO₄⁻ | 0.001586 | 0.000167 | 9.6 | 7.54 |
| 90. | III | 0.1 | H₂PO₄⁻ | 0.01586 | 0.00167 | 0.96 | 7.36 |
| 91. | III | 0.1 | H₂PO₄⁻ | 0.1586 | 0.0167 | 0.096 | 6.48 |
| 92. | III | 1.0 | H₂PO₄⁻ | 0.001586 | 0.000167 | 96 | 7.72 |
| 93. | III | 1.0 | H₂PO₄⁻ | 0.01586 | 0.00167 | 9.6 | 7.42 |
| 94. | III | 1.0 | H₂PO₄⁻ | 0.1586 | 0.0167 | 0.96 | 6.58 |
| 95. | III | 10.0 | H₂PO₄⁻ | 0.01586 | 0.00167 | 96 | 7.53 |
| 96. | III | 10.0 | H₂PO₄⁻ | 0.1586 | 0.0167 | 9.6 | 6.50 |
| 97. | III | | H₂PO₄⁻ | 1.586 | 0.167 | 0.96 | 6.14 |

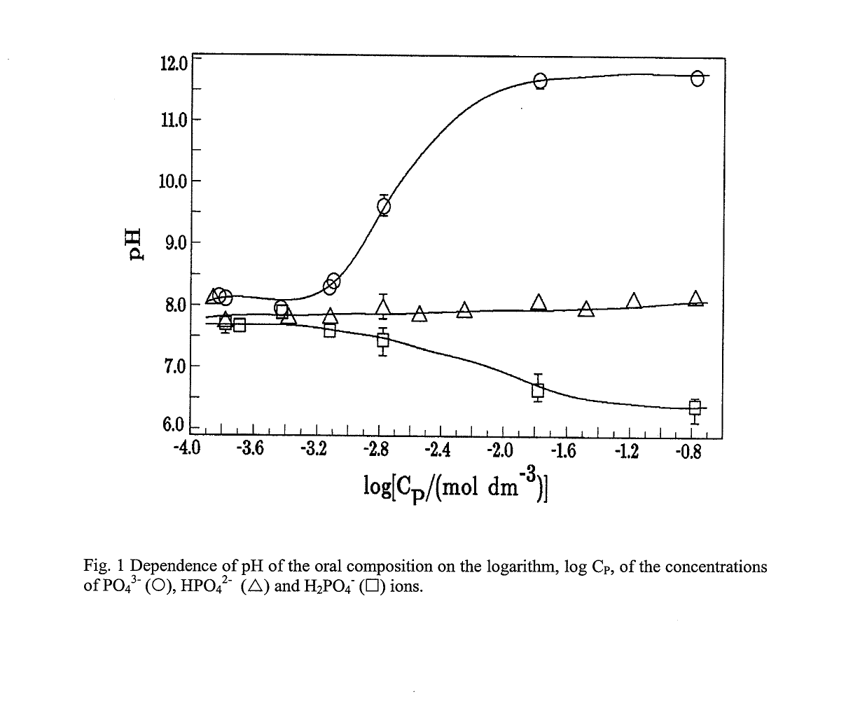

The results listed in Table 3 and represented in FIG. 1 show:
1. Mixing together water suspensions of a calcium form of zeolite and water solution of phosphate ions [e.g., addition of (Na,H)-phosphate salts into a water suspension of zeolite and/or addition of calcium form of zeolite into water solution of phosphate] considerably changes the pH of the mixture (see Table 3) relative to the water suspension of a calcium form of zeolite (see Table 1) and water solution of (Na,H)-phosphate (see Table 2) alone.
2. Although the presence of a calcium form of zeolite considerably influences the pH value of mixtures (calcium form of zeolite suspended in phosphate solution; see Tables 1-3), equilibrium pH value is determined by the type and concentration of the dissolved phosphate ions, but is not influenced by the type of zeolite and its content in the investigated range (0.1-10 wt. %; see Table 3 and FIG. 1)
3. pH of the suspension of the calcium form of zeolite(s) in the water solution(s) of Na₃PO₄ does not change significantly with the increase of concentrations of PO₄³⁻ ions in the concentration range from about 0.0001.5 mol dm⁻³ to about mol 0.0008 mol dm⁻³ (equilibrium pH is from about 8.12 to about 8.5 in the concentration range of PO₄³⁻ ions; see FIG. 1). At concentrations of PO₄³⁻ ions higher than 0.0008 mol dm⁻³, pH increases with their increasing concentration and reaches the maximum pH value of about 11.7 for concentrations of PO₄⁻ ions higher than 0.0167 mol dm⁻³.
4. pH of the suspension of the calcium form of zeolite(s) in the water solution(s) of Na₂HPO₄ changes little with the change of concentration of HPO₄⁻ ions; pH does change from about 7.80 to 8.11 when the concentration of HPO₄⁻ ions increased 0.00014 mol dm⁻³ to 0.167 mol dm⁻³ (see FIG. 1).
5. pH of the suspension of the calcium form of zeolite(s) in the water solution(s) of NaH₂PO₄ is approximately constant (about 7.7) for low concentrations of H₂PO₄⁻ ions (from about 0.00017 mol dm⁻³ to about 0.0008 mol dm⁻³), and progressively decreases with the increased concentration of H₂PO₄⁻ for the concentration of H₂PO₄⁻ higher than 0.0008 mol dm⁻³ and reaches the pH value of about 6.4 at concentrations of H₂PO₄⁻ ions of about 0.17 mol dm⁻³ (see Table 3 and FIG. 1).

The optimal pH (7.5-8) can be achieved by mixing a calcium form of zeolite (0.1-10 wt. %, regardless of the type) with 0.00015 mol dm⁻³ to about 0.0008 mol dm⁻³ solution of Na₃PO₄, or with 0.00014 mol dm⁻³ to 0.167 mol dm⁻³ solution of Na₂HPO₄, or with 0.00017 mol dm⁻³ to about 0.0008 mol dm⁻³ solution of NaH₂PO₄. As shown in FIG. 1, pH values higher than 8 can be obtained only with Na₃PO₄ for the concentrations of PO₄³⁻ ions higher than 0.0008 mol dm⁻³. At the same time, these results show that pH remains approximately constant when the systems containing PO₄³⁻ and H₂PO₄⁻ are diluted by a factor of 5, and when the system containing HPO₄²⁻ ions is diluted by a factor of 1200.

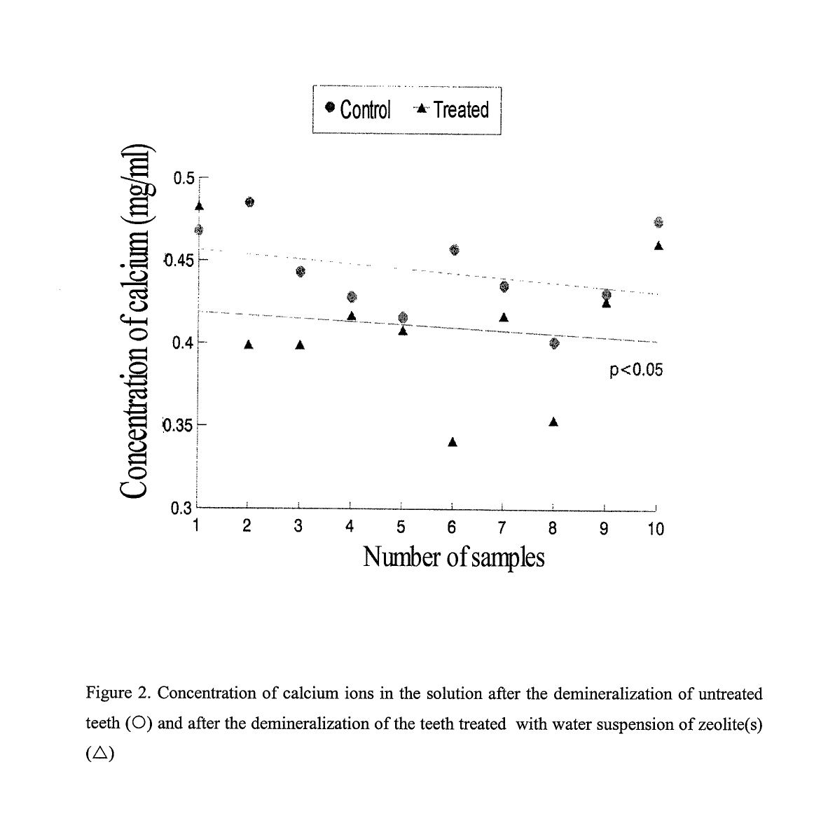

To study the influence of pH on the process of demineralization and stability of teeth, a sample of 15 teeth has been tested using the suspensions of a calcium form of zeolite as a regulator of pH, and as a source of calcium ions. Each sample of teeth was divided into two parts. One part of each tooth was treated for 9 minutes in a suspension which contained 1 g of the calcium form of zeolite of the type I in 10 (5 teeth) and 100 ml of demineralized water (5 teeth), or of the type II in the 10 ml of demineralized water (5 teeth) (pH=8.02-9.05), respectively. Thereafter, the teeth were washed with demineralized water, and dried. Dried treated and untreated parts of teeth were demineralized for 12 hours in a 4 M (pH=3.5) solution of acetic acid at 37° C. in dynamic conditions (mixing the suspension with samples of teeth). After the process of demineralization was finished, the concentration of calcium ions in the control group (untreated teeth), generally was about 0.42 to 0.5 mg/ml while the concentration of calcium ions in the groups of teeth treated with the water suspension of zeolite(s) statistically decreased about 10%. FIG. 2 represents the concentration of calcium ions in the solution after the demineralization of untreated teeth and after demineralization of teeth treated with water suspension of zeolite(s).

The results obtained can be explained by remineralization (recalcification) of dental enamel and simultaneous stabilization during the treatment in the alkaline suspension of calcium form of zeolite and accordingly, slower process of demineralization in the acidic medium (0.4 M acetic acid; pH=3.5).

In order to prove the conclusion resulted from the investigation of the influence of pH on the stability of teeth, samples of teeth were treated with solutions prepared in the following way: (i) solution (L-Ca)o was prepared by a centrifugal separation of the solid phase (calcium form of zeolite of type I) from the suspension (1 g of the calcium form of zeolite in the 100 ml demineralized water; pH 8.01), stirred for 24 hours at room temperature and; (ii) solution (L-Na)o was prepared by a centrifugal separation of the solid phase (sodium form of zeolite of type I) from the suspension (1 g of the sodium form of zeolite in the 100 ml demineralized water; pH=10.5), stirred for 24 hours at room temperature. The solution (L-Ca)₀ contained 0.01 mg Ca²⁺ ions/cm³ (see Table 4) as a consequence of equilibrium of the process of substitution of H⁺ ions from the water according to the equation:

CaZ+2H₂O<=>H₂Z+Ca²⁺+2OH⁻ (1)

Each of tooth (samples S₁-S₄ for treatment with solution (L-Ca)o and samples S₅-S₇ for the treatment with solution (L-Na)₀ was divided into two approximately equal parts. One part of teeth, was treated with solution (L-Ca)₀ or (L-Na)₀ (12 hours at 37° C. under stirring). The second part of the teeth was, under the same conditions (12 hours at 37° C. under stirring), treated with 0.4 M solution of acetic acid (solution K). After treatment, the concentration of Ca²⁺ ions in the solution was determined by atomic absorption spectroscopy (AAS). The results are shown in Table 2 as the concentrations of Ca²⁺ ions in the solutions (L-Ca)₁-(L-Ca)₄ obtained after the treatment of the samples S₁-S₄ with the solution (L-Ca)₀, in the solutions (L-Na)₅-(L-Na)₇ obtained after treatment of the samples S5-S7 with the solution (L-Na)₀ and in the solutions K₁-K₇, obtained by treatment of the second parts of the samples S₁-S₇ with 0.4 M solution of acetic acid (solution K).

TABLE 4
The influence of the mode of treatment of teeth on the
concentration of Ca²⁺ ions in the solutions

| SAMPLE | CONCENTRATION OF Ca²⁺ (mg/cm³) |
|---|---|
| (L-Ca)₀ | 0.01 |
| (L-Na)₀ | 0.0013 |
| (L-Ca)₁ | 0.0017 |
| K₁ | 3.446 |
| (L-Ca)₂ | 0.0022 |
| K₂ | 4.052 |
| (L-Ca)₃ | 0.0029 |
| K₃ | 2.430 |
| (L-Ca)₄ | 0.0036 |
| K₄ | 3.84 |
| (L-Na)₅ | 0.0012 |
| K₅ | 3.187 |
| (L-Na)₆ | 0.00029 |
| K₆ | 3.516 |
| (L-Na)₇ | 0.00092 |
| K₇ | 1.995 |

The results presented in Table 4 show the concentrations of calcium in alkaline solutions (L-Ca)₁-(L-Ca)₄ are approximately 1440 times lower than in the acidic solutions K₁-K₄, and that the concentrations of calcium in the alkaline solutions (L-Na)₅-(L-Na)₇ are approximately 5600 times lower than in the acidic solutions K₅-K₇, after contact with the samples of teeth. This means that the decalcification in the alkaline solutions L-Ca and L-Na has been reduced more than 3 three orders of magnitude (at pH=8.01) or more (at pH=10.5) relative to decalcification in the acidic solutions K (pH=3.5). Although the greater effect of stabilization at a higher pH value (solution (L-Na)₀; pH=10.5) was expected, the reduction of the concentration of calcium in the solution (L-Ca)₀ from the starting value of 0.01 mg/cm³ to approximately 0.0026 mg/dm³ clearly indicates that about 75% of calcium ions from the solution (L-Ca)₀ was incorporated in the dental enamel during treatment. The alkaline environment established by the presence of the calcium form of zeolite not only significantly lowers the process of decalcification, but also stimulates the process of calcification.

In order to prove the process of (re)calcification during treatment and to separate the influence of stabilization of dental enamel in the alkali environment from the influence of (re)calcification on the lowering of demineralization in the acidic medium, samples of teeth were been divided into two approximately equal parts, and thereafter the samples were treated as follows:

Series A (3 samples): one half of each of three teeth from the series A was demineralized in an acidic medium (0.4 M solution acetic acid) under dynamic conditions (stirring) at 37° C. for 6 hours. Another half of each of three teeth was treated with the suspension containing 1 g of the calcium form of zeolite of the type I in 100 ml of demineralized water, under dynamic conditions (stirring) at 37° for 60 min, before the demineralization. Series B (3 samples): one half of each of three teeth from the series B is demineralized for 6 hours in the acidic medium in the same way as the samples from the series A. Another half of each of three teeth was treated with the suspension contained 1 g of sodium form of zeolite of the type I in 100 ml of demineralized water, under dynamic condition (stirring) at 37° for 60 min, before the demineralization.

TABLE 5
The influence of incorporation of calcium ions
in the enamel on the stabilization of teeth

| Sample No.: | Concentration of Ca²⁺ (mg/ml) | X+ (%) | 100 − X+ (%) |
|---|---|---|---|
| A₁ | 0.744 | — | — |
| A₁-(Ca) | 0.582 | | 25 |
| A₂ | 0.642 | — | — |
| A₂-(Ca) | 0.556 | 86.6 | 13.4 |
| A₃ | 0.632 | — | — |
| A₃-(Ca) | 0.506 | 80 | 20 |
| B₁ | 0.788 | — | — |
| B₁-(Na) | 0.582 | 88 | 12 |
| B₂ | 0.655 | — | — |
| B₂-(Na) | 0.595 | 91 | 9 |
| B₃ | 0.620 | — | — |
| B₃-(Na) | 0.631 | 101.8 | −1.8 |

The concentrations of calcium ions in the solutions after decalcification in the acidic medium are shown in Table 5. The designations A₁-A₃ correspond to the solutions obtained after decalcification of the teeth from the series A. The designations B₁-B₃ correspond to the solutions obtained after decalcification of the teeth from the series B. The designations A₁-(Ca)-A₃-(Ca) correspond to the solutions obtained after decalcification of the teeth from the series A, which were previously treated with the suspension containing 1 g of the calcium form of zeolite of type I in 100 ml of demineralized water. The designations B₁-(Na)-B₃-(Na) correspond to the solutions obtained after decalcification of the teeth from the series B, which were previously treated with the suspension contained 1 g of the sodium form of zeolite of type I in 100 ml of demineralized water. The meaning of X+ is the percentage of the concentration of calcium ions in alkaline solution relative to the concentration of calcium ions in the corresponding acidic solution, e.g., X+=75% means that the concentration of calcium ions in alkaline solution is 75 of the concentration of calcium ions in the acidic solution, or in the other words, that the concentration of calcium ions in the alkaline solution is 25% (=100−X+) lower than in the corresponding acidic solution.

The concentrations of calcium in the solutions A-(Ca)ₙ and B-(Na)ₙ are 9-25 lower than in the solutions Aₙ and Bₙ. However, the average lowering in the concentration of calcium ions in the solutions A-(Ca)ₙ is about 19.5%, relative to the concentrations of calcium ions in the solutions Aₙ, while the average lowering in the concentration of calcium ions in the solutions B-(Na)ₙ is about 6.4% relative to the concentrations of calcium ions in the solutions Bₙ. Hence one can conclude that the increased level of stabilization of teeth in the suspension of calcium forms of zeolite in comparison with the suspension of sodium form of zeolite is caused by the incorporation of calcium from the calcium form of zeolite into the dental enamel (calcification) during the treatment.

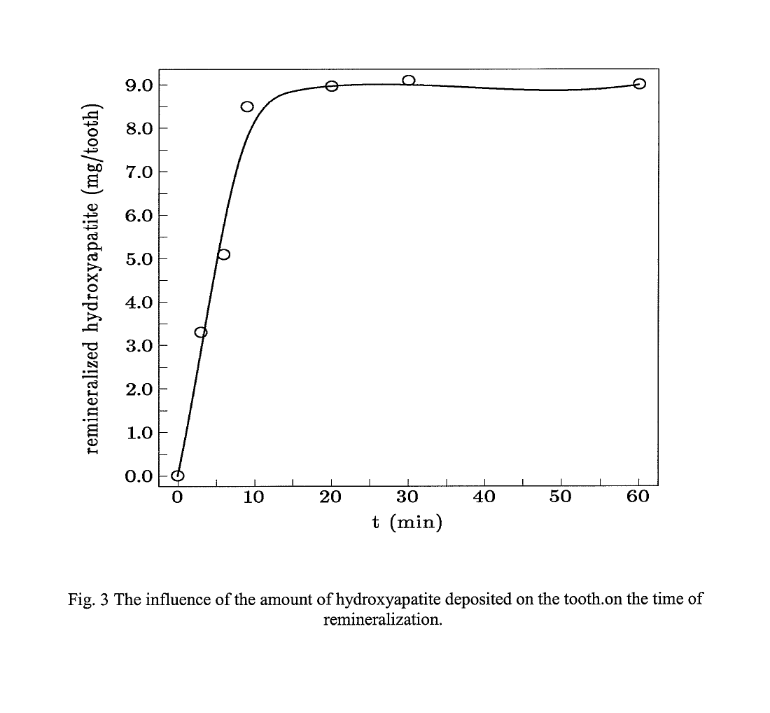

In order to evaluate the assumption about the simultaneous incorporation of calcium and phosphate ions into the dental enamel and dentin and the formation of hydroxyapatite (remineralization), samples of teeth were treated with the solution which contained calcium and phosphate ions. The solution was prepared by separation of clear liquid phase from the freshly prepared oral composition which contained 1 g calcium form of zeolite of the type I in the 100 ml of 5×104 M Na₂HPO₄ solution. Such prepared solution has pH 7.8 and contained 3.1×10⁻⁴ mol dm⁻³ phosphate ions and 1.2×10⁻⁴ mol dm⁻³ of calcium ions. The solution is divided into 5 equal aliquots of 10 ml each. In each aliquot 10 ml of solution mixed with magnetic mixer is put one tooth having approximately the same mass. The moment of placing the tooth into the solution is used as zero time (t=0) of the process of remineralization. In predetermined times, t, after the beginning of the process of remineralization, aliquot samples (1 ml) were drawn of the solution in order to measure the concentrations of calcium and phosphate ions. The amounts of calcium and phosphate ions, expressed as the amount of hydroxyapatite incorporated in the tooth (see FIG. 3), have been calculated from the difference between the initial concentrations of calcium and phosphate ions in the solution and the concentrations of the same ions after certain time of remineralization. The results are shown in FIG. 3, as average amounts of hydroxyapatite deposited (built up) on tooth at different times of remineralization. The amount of hydroxyapatite deposited on tooth is a linear function of time of remineralization; in the above mentioned example, the process is finished in approximately 10 min.

It is important to emphasize that the rate of remineralization and the amount of deposited hydroxyapatite can be adjusted with the concentrations of phosphate and calcium form of zeolite in the suspension. It is especially important that the excess of one of the components (calcium or phosphate ions) does not change the chemical composition of deposited hydroxyapatite, i.e., that the amount of deposited hydroxyapatite is determined by the concentration of component that is in deficit (i.e., by the concentration of calcium ions in the mentioned case).

Based on the presented results, we conclude that the decreased dissolution of tooth enamel and dentin (demineralization, expressed as the concentration of calcium and/or phosphate ions), of teeth treated with the oral composition, relative to the untreated teeth, can be ascribed to the stabilization of mineral portion of tooth (enamel and dentin) by the activity of an excess of OH⁻ ions in the mildly alkaline environment and by simultaneous incorporation of calcium and phosphorus from the oral composition into the enamel and dentin (remineralization). Since demineralization is a time dependent process and demineralization and (re)remineralization are parallel processes, it can be assumed that the time of treatment of teeth with the oral composition significantly influences effects of stabilization, (re)calcification and (re)mineralization of teeth.

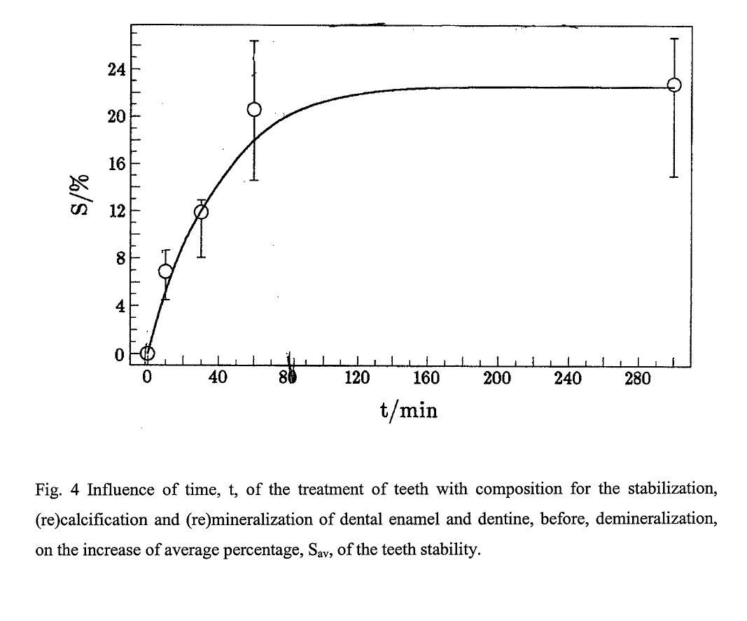

The results of testing of an influence of the time of treatment of teeth with the oral composition according to the invention are shown on FIG. 4. Depending on the cumulative time of treatment (10-300 minutes), the stability of dental enamel and dentin increases from 7 to 29% with respect to the untreated teeth (see FIG. 4). The results in FIG. 4 show that the value S % increases with treatment time; S % reached the maximum value (>23%) in approximately 60 minutes, and further treatment does not have a significant effect. Therefore, one can conclude that the reduction of demineralization after treatment with oral composition is caused by deposition of hydroxyapatite on the surface of tooth enamel (remineralization), and by stabilization of the newly formed enamel under optimal pH conditions. It was also shown that an addition of enamel matrix protein in the oral composition increases the rate of remineralization, and at the same time increases the tooth stability 30-50% relative to the oral composition without enamel matrix protein.

### EXAMPLE 1

Enamel matrix protein (EMP) is a component of mineralized tissues such as bone, dentin, cementum and calcified gristly. Enamel matrix protein is a significant component of the extracellular bone matrix and has been suggested to constitute approximately 8% of all non-collagenous proteins found in bone and cementum. Enamel matrix protein was originally isolated from the bovine cortical bone (powder) as a 23-kDa glycopeptide with high sialic acid content, as described in separate reports in Biochim. Biophys. Acta. 1965 101:327-35. Shortly modified protocol: Purification of enamel matrix protein isolated from bone powder was achieved by ion exchange chromatography on a DEAE-cellulose column. The eluting buffer for isolation of enamel matrix protein was 50 mM of sodium acetate containing 7 M urea and 0.5% (wt/vol) Triton X-100 at pH 6.0. After digestion of bone powder with 7 M urea overnight (o/n) aliquots of 10 ml was dialysis against PBS o/n and lyophilized. Powder was resuspended in 0.05 M potassium acetate buffer pH 5.0. After equilibration of the DEAE columns by washing with a buffer, samples of the fractions were applied in a 0.05 M-acetate buffer, pH 5-0. Elution with a flow rate of about 10 ml/hr was carried out either with a linear gradient of 0.0-1.0 M NaCl in 0.05 M-acetate buffer, pH 5.0. One-minute fractions were collected during a total run time of 60 min, respectively. Fractions were collected between 0.6 to 0.9 M of NaCl of linear gradient and then dialysis against PBS o/n at +40C and lyophilized. The final product contains 1.32×10⁻⁴−0.1 wt. % of enamel matrix proteins. Enamel matrix protein in these examples was upregulated process of dentin mineralization after 7 days of therapy by our composition.
The term “upregulated” as used herein refers to the process by which the number of components increases in response to external variables.

In one embodiment of the invention, in the application of oral composition the components of the oral composition are physically separated. The above mentioned physical separation can be implemented: (a) using chambers separated by impermeable barrier in the tube (toothpaste), (b) via microencapsulation of one of the components (toothpaste, chewing gum, bonbons, gel) or (c) mixing the components of oral composition into different layers (chewing gum, bonbon).

The oral composition according to the invention can be used in the form of a toothpaste, dental paste, chewing gum, gel, mouth rinse , bonbons and others preparations that stay in the mouth cavity.
When the invention is used in the form of toothpaste, in accordance with the invention, the components are present in the following amounts:

| | | |
|---|---|---|
| 1. Calcium form of zeolite: | 0.1-10 | wt. % |
| 2. Phosphate ions: | 0.00132-2 | wt. % |
| 3. Matrix proteins: | 1.32 × 10⁻⁴-0.1 | wt. % |

4. Except the composition according to the invention, the toothpaste can contain:

Abrasives such as silicon dioxide in different forms, aluminum hydroxide, aluminum oxide or mixtures thereof.

Thickening agents such as glycerin, propylene glycol, polyethylene glycol, mannitol, sorbitol, mineral oils, vegetable oils or mixtures thereof.

Binding agents or stabilizers such as synthetic polymers soluble in water, agar-agar, pectins, carboxyl methylcellulose, xanthic rubber, carboxyl vinyl polymers, polyvinyl alcohol, polyvinyl pyroles, carogenane, tragacanth gum, rubber, guar, cellulose, methyl hydroxypropyl cellulose or mixtures thereof.

Surface active substances (foaming agents) such as sodium-N-lauryl sarcosinate, sodium lauryl sulfate, palm oil, coconut oil or mixtures thereof.

Sweeteners such as sodium saccharin, sodium cyclamate, sorbitol, xylitol, lactose, maltose, fructose or mixtures thereof.

Corigenses of taste such as mint oil, spearmint oil, chamomile oil, sage oil, eucalyptus oil, the oil of tea plant, thyme oil, cinnamon oil, fennel oil, cardamom oil or mixtures thereof.

Solvents such as lower polyhydroxylated alcohols and ethers or mixtures thereof. The given examples of abrasives, thickening agents, bonding agents, surface acting agents, sweeteners, corigenses of taste and solvents in no way limit all possible substances that can be used for same purpose.

When the invention is used in the form of chewing gum, in accordance with the invention, the components are present in the following amounts:

| | | |
|---|---|---|
| 1. Calcium form of zeolite: | 0.1-10 | wt. |
| 2. Phosphate ions: | 0.00132-2 | wt. |
| 3. Matrix proteins: | 1.32 × 10⁻⁴-0.1 | wt. |

4. Standard gum-bases, standard plasticizers, standard sweeteners, standard eastemers, standard filling agents, standard softeners, standard emulsifying agents, standard colors and standard aromas.

Only some specific implementations of this innovation are presented in this patent application. The professionals in this area know that there are various versions of this innovation are possible. It must be emphasized that all such kinds of realizations and implementations of this innovation are included within the patent applications which follow.

1. An oral composition for stabilization, recalcification and remineralization of dental enamel and dentin comprising: a calcium form of zeolite selected from the group consisting of type I, II and III, a phosphate ion source, and enamel matrix proteins, wherein the pH of said oral composition is between from about 7.5 to about 11.9.

2. The oral composition for stabilization, recalcification and remineralization of dental enamel and dentin as in claim 1, wherein the calcium form of zeolite selected from the group consisting of type I, II and III comprises 0.1-10 wt. % of the composition.

3. The oral composition for stabilization, recalcification and remineralization of dental enamel and dentin as in claim 1, wherein the phosphate ion source is a water soluble sodium phosphate selected from the group consisting of Na₃PO₄, Na₂HPO₄ and NaH₂PO₄, preferably, Na₂HPO₄.

4. The oral composition for stabilization, recalcification and remineralization of dental enamel and dentin as in claim 3, wherein the water soluble sodium phosphate is Na₂HPO₄.

5. The oral composition for stabilization, recalcification and remineralization of dental enamel and dentin as in claim 1, wherein the phosphate ions comprise between 0.00132-1.586 wt. % of the composition.

6. The oral composition for stabilization, recalcification and remineralization of dental enamel and dentin as in claim 1, wherein the molar ratio of calcium to phosphate (Ca/P) is from 0 to about 170.5.

7. The oral composition for stabilization, recalcification and remineralization of dental enamel and dentin as in claim 1, wherein the enamel matrix proteins comprise between 1.32×10⁻⁴−0.1 wt. % of the composition.

8. The oral composition for stabilization, recalcification and remineralization of dental enamel and dentin as in claim 2, further comprising: 0.1-10 wt. % of the calcium form of zeolite; 0.00132-2 wt. % of phosphate ions; 1.32×10⁻⁴−0.1 wt. % of enamel matrix proteins; and one or more additional ingredients selected from the group consisting of: abrasives, thickening agents, binding agents, surface acting agents, sweeteners, corigenses of taste, solvents, and mixtures thereof.

9. The oral composition for stabilization, recalcification and remineralization of dental enamel and dentin as in claim 8, further comprising between 0.01-0.025 wt. % of an milfoil extract and/or oil.

10. The oral composition for stabilization, recalcification and remineralization of dental enamel and dentin as in claim 1, wherein release of calcium ions from the calcium form of zeolite is controlled release.

11. The oral composition for stabilization, recalcification and remineralization of dental enamel and dentin as in claim 1, wherein a controlled reaction between the released calcium ions and dissolved phosphate ions occurs.

12. The oral composition for stabilization, recalcification and remineralization of dental enamel and dentin as in claim 11, wherein the controlled reaction of calcium and phosphate ions occurs on a tooth surface.

13. The oral composition for stabilization, recalcification and remineralization of dental enamel and dentin as in claim 1, wherein the enamel matrix proteins increase remineralization rates.

14. The oral composition for stabilization, recalcification and remineralization of dental enamel and dentin as in claim 1, wherein pH of the oral composition ranges between about 4.88 to about 11.82

15. The oral composition for stabilization, recalcification and remineralization of dental enamel and dentin as in claim 14, wherein pH of the oral composition ranges between about 8.12 to about 8.50.

16. The oral composition for stabilization, recalcification and remineralization of dental enamel and dentin as in claim 15, wherein the pH of the oral composition is unaffected by dilution.

17. The oral composition for stabilization, recalcification and remineralization of dental enamel and dentin as in claim 15, wherein the pH of the oral composition between about 4.88 to about 11.82 stabilizes hydroxyapatite formed on teeth during remineralization.

18. The oral composition for stabilization, recalcification and remineralization of dental enamel and dentin as in claim 1, wherein the enamel matrix proteins increase stability of hydroxyapatite formed on teeth during remineralization about 30-50%.

19. The oral composition for stabilization, recalcification and remineralization of dental enamel and dentin as in claim 1, wherein the composition is selected from the group consisting of a toothpaste, chewing gum, bonbons, candy, mouth rinse, film, and lozenge.

20. The oral composition for stabilization, recalcification and remineralization of dental enamel and dentin as in claim 19, wherein the composition is a toothpaste.

21. The oral composition for stabilization, recalcification and remineralization of dental enamel and dentin as in claim 1, wherein the calcium ion source, phosphate ion source, and enamel matrix proteins are physically separated.

22. The oral composition for stabilization, recalcification and remineralization of dental enamel and dentin as in claim 1, wherein the calcium ion source, phosphate ion source, and enamel matrix proteins are located in different layers.

23. The oral composition for stabilization, recalcification and remineralization of dental enamel and dentin as in claim 1, wherein the calcium ion source, phosphate ion source, and enamel matrix proteins are physically separated by micro encapsulation.

24. A process for stabilization, recalcification and remineralization of dental enamel and dentin comprising:
application to teeth of a composition comprising a calcium ion source, a phosphate ion source, and enamel matrix proteins; wherein the calcium form of zeolite comprises the calcium ion source.

25. The process of claim 24, wherein increased level of stabilization of teeth results from the incorporation of calcium from the calcium form of zeolite into dental enamel.

26. The process for stabilization, recalcification and remineralization of dental enamel and dentin comprising application of a composition as in claim 24 for specific requirements selected from the group consisting of older persons, children, osteoporosis, gingivitis prophylaxis and gingivitis treatment.

27. A dental kit for stabilization, recalcification and remineralization of dental enamel and dentin comprising: an oral composition for stabilization, recalcification and remineralization of dental enamel and dentin comprising: a calcium ion source, a phosphate ion source, and enamel matrix proteins together with instructions for use.
